(19) United States
(12) Patent Application Publication
(10) Pub. No.: US 2015/0106124 A1
(43) Pub. Date: Apr. 16, 2015

(54) DATE AND TIME ACCURACY TESTING PATIENT DATA TRANSFERRED FROM A REMOTE DEVICE

(71) Applicant: Medtronic, Inc, Minneapolis, MN (US)

(72) Inventors: Daniel L. COSENTINO, Excelsior, MN (US); Brian A. Golden, Eden Prairie, MN (US)

(73) Assignee: Medtronic, Inc, Minneapolis, MN (US)

(21) Appl. No.: 14/514,850

(22) Filed: Oct. 15, 2014

Related U.S. Application Data
(60) Provisional application No. 61/891,152, filed on Oct. 15, 2013.

(51) Int. Cl. A61B 5/00; G06F 19/00

(52) U.S. Cl. CPC A61B 5/7221; G06F 19/322
USPC 705/3

(57) ABSTRACT

A system and method for ensuring the accuracy of health related information. This system is used for receiving, processing, analyzing, and verifying the accuracy of healthcare data prior to its transmission to a client. Embodiment described may enable patient data, collected from various patient input devices, to be automatically filtered or categorized into one of several categories. The categories are based on the potential accuracy of the data, which are determined by a set of pre-determined rules. Data that surpasses accuracy testing may then be transmitted to a client (hospitals, patient physicians, patient caregivers, patients, etc.) for review and/or incorporation into client records. Data that fails accuracy testing may be discarded.

### CROSS-REFERENCE TO RELATED APPLICATION

This application claims priority to U.S. Provisional Application No. 61/891,152, filed on Oct. 15, 2013, which is incorporated herein in its entirety by reference.

### INTRODUCTION

Caregivers frequently wish to review a wide range of patient health information to determine a patient's health status. Meeting with a patient to gather this information (e.g., patient measurements, biometrics, and the like) requires resources such as time and money for both the caregiver and the patient. Receiving patient medical information from a remote patient is more convenient. However, massive quantities of patient information may be received from each patient. Further, it is difficult to assess the quality and accuracy of the received patient information.

Devices used to gather patient data may have inaccurate or incorrect date and time at the time a measurement is taken. It is especially important to ensure that the patient information have accurate date and time settings. For example, it would be important to know whether the weight is measured in the morning, afternoon, or evening, whether a glucose reading is before or after a meal, whether a blood pressure measurement is a function of an environmental event, whether a medication is taken at a specific time of the day, and when a stressful event takes place. There are many other examples, especially those related to medication dosing and to vital sign measurements, for which date and time information is integral to the clinical interpretation thereof. Many different problems may occur if such date and time settings are inaccurate. For example, if inaccurate patient information is included with accurate information in a treaded graphical display of a patient's historical data, the display can be difficult or impossible for the viewer to interpret correctly. Similarly, if inaccurate measurements are included in automated trend calculations, errors or incorrect results may occur, requiring manual inspection of each measurement in the data set in order to identify and remove the inaccurate measurements.

### SUMMARY

In general terms, this disclosure is directed to systems and methods for ensuring accuracy of one or more patient readings from one or more patient monitoring devices. The received data is analyzed for accuracy based on one or more comparison analyses of the time and date settings.

It should be appreciated that aspects of the above-described subject matter may be implemented as a computer-controlled apparatus, a computer process, a computing system, a network of communicating computing systems, or as an article of manufacture such as a computer-readable storage medium. These and various other features will be apparent from a reading of the following Detailed Description and a review of the associated drawings.

This disclosure is provided to introduce a selection of concepts in a simplified form that are further described below in the Detailed Description. This disclosure is not intended to identify key features or essential features of the claimed subject matter, nor is it intended that this be used to limit the scope of the claimed subject matter. Furthermore, the claimed subject matter is not limited to implementations that solve any or all disadvantages noted in any part of this disclosure.

### BRIEF DESCRIPTION OF THE DRAWINGS

Reference will now be made to the accompanying drawings, which are not necessarily drawn to scale and wherein:

FIG. 1 is a block diagram illustrating an exemplary embodiment of a segregation system.

FIG. 2 is a schematic block diagram illustrating an exemplary architecture of a computing device for implementing aspects of the system shown in FIG. 1.

FIG. 3 is a flow chart illustrating an exemplary embodiment of a method of analyzing the time accuracy of patient readings.

FIG. 4 is a flow chart illustrating an exemplary embodiment of a method of comparison analysis.

FIG. 5 is a flow chart illustrating an exemplary embodiment of a method of comparison analysis.

FIG. 6 is an embodiment of a general method 600 for assigning a probability of accuracy to an individual measurement received from a device.

### DETAILED DESCRIPTION

In general, the present disclosure describes systems and methods for ensuring the accuracy of health related information. These systems are used for receiving, processing, analyzing, and verifying the accuracy of healthcare data prior to its transmission to a client. For example, embodiments described below may enable patient data, collected from various patient input devices, to be automatically filtered or categorized into one of several categories. The categories are based on the potential accuracy of the data, which are determined by a set of pre-determined rules. Data that surpasses accuracy testing may then be transmitted to a client (hospitals, patient physicians, patient caregivers, patients, etc.) for review and/or incorporation into client records. Data that fails accuracy testing may be discarded or may be transmitted to a client or another system for further analysis or display.

It should be noted here that by “time accuracy,” “accuracy,” and “probability of accuracy” of measurements and readings, it is meant that the date and time of those measurements are likely to be accurate or not. That is, accuracy does not refer to the how close the measurement is to the true value, but rather the accuracy of the date and time associated with the measurement. For example, a heart rate monitor may take an correct measurement of a patient's heart rate as 66 beats per minute, but assign the measurement a date and time of 4:15 Greenwich Mean Time, in the year 455 AD. In this case, the systems and methods described herein should identify this measurement as having a likelihood of being highly inaccurate, or having a low accuracy, so that the measurement/date/time combination can be flagged and not displayed to health care professional along with accurate measurements or otherwise used with accurate measurements in time sensitive analyses.

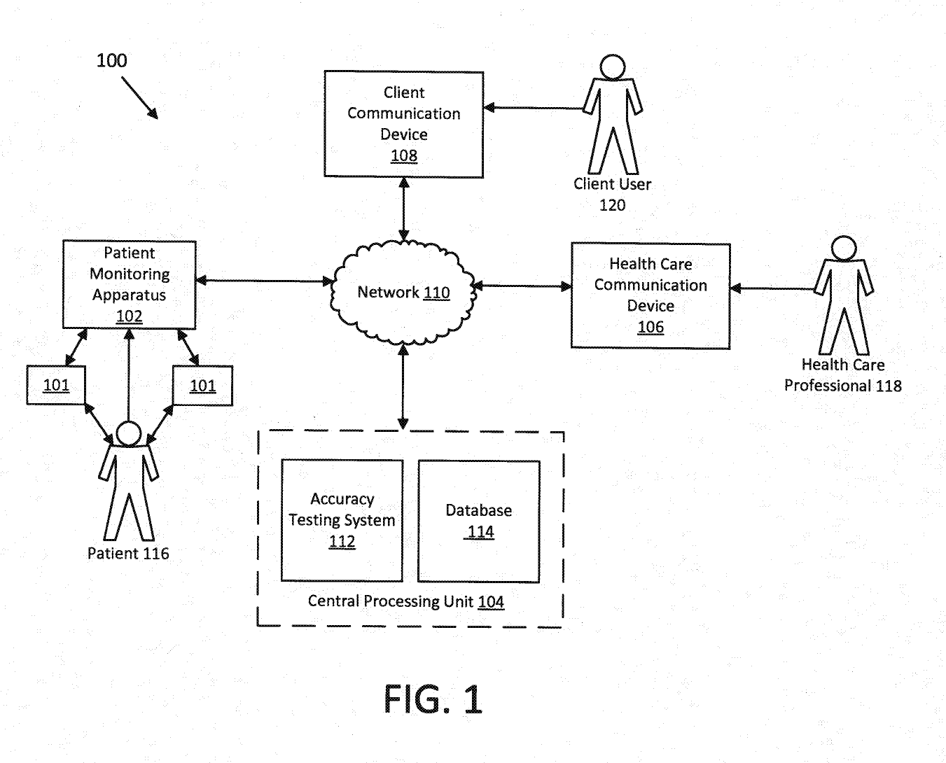

FIG. 1 is an embodiment of a system 100 for testing the accuracy of healthcare data. More specifically, as shown in the FIG. 1, the system 100 can be used for reviewing the accuracy of incoming patient readings. For example, the system 100 includes a patient monitoring apparatus 102, a central processing unit 104, a healthcare communication device 106, a client communication device 108, and one or more networks 110. A patient 116, a healthcare professional 118, and a client user 120 may access the patient monitoring apparatus 102, the healthcare communication device 106, and the client communication device 108, respectively. Depending on the implementation, the healthcare profession 118 and client user 120 may also have limited or complete access to any of the devices on the network.

The patient monitoring apparatus 102 collects data from the patient and transmits it to one or more of the other components of the system 100. The patient data, which may also be referred to as patient information, may be stored in the database 114 until it is reviewed by the health care profession 118 and/or transmitted to the client communication device 108.

The patient data are tested by an accuracy testing system 112 to determine the accuracy of the data. As will be described in greater detail below, the testing may result in patient data being categorized based on accuracy. For example, data may be categorized as highly accurate or highly inaccurate based on various analyses performed by the accuracy testing system 112. In the embodiment shown, the central processing unit 104 includes an accuracy testing system 112 and a database 114. In an alternate embodiment of the system 100, it is understood that the accuracy testing system 112 may be contained within the patient monitoring apparatus 102 instead of the central processing unit 104. It is understood that such a system generally operates in the same or similar ways as described below, unless distinguished. In different embodiments, the accuracy testing system 112 may be implemented as a separate and discrete device, as a separate application or program executing on the data, or as subroutines or sub programs within a larger processing system or executing program on a computing device as a matter of design choice.

Each of the patient monitoring apparatus 102, the client communication device 108, and the health care communication device 106 is capable of directly and/or indirectly communicating with the central processing unit 104 across one or more networks 110. The networks 110 can comprise any of a number of different combinations of one or more different types of networks. For example, the networks 110 can include one or more data private and/or public networks, such as a local area network (LAN), a metropolitan area network (MAN), and/or a wide area network (WAN) (e.g., Internet), can include one or more wireline and/or wireless voice networks such as a public-switched telephone network (PSTN), and/or wireless networks. For purposes of illustration and simplicity, the network 110 comprises wireless connection of the patient monitoring apparatus 102 to the Internet (i.e., WAN) unless otherwise noted.

The patient monitoring apparatus 102, the central processing unit 104, the healthcare communication device 106, and the client communication device 108 can comprise any one or more of a number of different entities, devices, or the like capable of operating in accordance with embodiments described herein. In this regard, one or more of the patient monitoring apparatus 102, the central processing unit 104, the healthcare communication device 106, and the client communication device 108 can comprise, include, or be embodied in one or more processing elements, such as one or more of a laptop computer, desktop computer, server computer or the like. Additionally or alternatively, one or more of the patient monitoring apparatus 102, the central processing unit 104, the healthcare communication device 106, and the client communication device 108 can comprise, include, or be embodied in one or more portable electronic devices, such as mobile computing device, smart telephone (e.g., iPhone), portable digital assistant (PDA), electronic tablet, pager, tablet computer, laptop computer, or the like. For example, the patient monitoring apparatus 102, the central processing unit 104, the healthcare communication device 106, and the client communication device 108 can each comprise a processing element capable of communicating with one another across the network 110.

The patient monitoring apparatus 102 may be located in the home of an ambulatory patient, or in some other location that is easily accessible to the patient 116. Generally, the patient monitoring apparatus 102 may be any device capable of receiving patient healthcare information. Alternatively, the patient monitoring apparatus 102 may be any device capable of sensing patient healthcare information, automatically or manually. In one embodiment, the patient monitoring apparatus 102 is configured to monitor and read wellness parameters of the patient 116, and then transmit the information as data to the central processing unit 104. Examples of patient data include blood pressure measurements, blood glucose measurements, electrocardiograph (ECG) measurements, weight measurements, pulse oxygen, blood oxygen measurements, pain, mood, heart rate, heart rhythm, peak flow, and any other measurements or biometrics. In addition, patient healthcare data may include patient answers to health-related questions.

Although not shown in the figure, it is understood that more than one patient monitoring apparatus 102 may be present in the system 100. The patient monitoring apparatus 102 may be any device capable of receiving health data from a patient. The patient monitoring apparatus 102 allows information to be obtained from the patient through direct interaction with the patient using question and answer sessions, such as by means of a video display and input system and an interactive voice recognition (“IVR”) system by which a patient may interact with the system. The patient monitoring apparatus 102 may include or be implemented using some or all of a telephone, modem, cell phone, tablet, any other computer, or the like. It is further understood that the system may monitor a plurality of patients. Each of the plurality of patients may interact with one or more monitoring devices which transmit data to the central processing unit 104.

In the embodiment shown, in addition to receiving data or inputs directly from the patient 116, the patient monitoring apparatus can also receive data from any of the attached monitoring peripherals 101. Examples of monitoring peripherals include any measurement collecting or monitoring device (e.g., blood pressure cuff, glucose meter, heart rate monitor, video capture device, weight scale, ECG, etc.).

The received patient information may be stored in the database 114 until it is reviewed by the health care professional 118 and/or transmitted to the client communication device 108. Alternatively, some patient data may be automatically discarded based on the accuracy testing, either by the central processing unit 104 or the patient monitoring apparatus 102. The healthcare professional 118 may directly access the patient data stored in the database 114 or indirectly access the patient data by utilizing the healthcare communication device 106 to connect to the central processing unit 104 via the network 110. The healthcare professional 118 may then access some or all of the patient information stored in the database 114 to review, verify, and/or modify the categorizations of the patient information. The patient information that is reviewed and verified by the healthcare professional 118 is then transmitted to the client communication device 108. In other embodiments, data that is reviewed by the accuracy testing system 112 and marked as highly inaccurate may be immediately discarded. Likewise, data that is marked as highly accurate may be immediately sent to the client communication device 108 without review by the healthcare professional 118. Data that is considered inaccurate by the accuracy testing system 112 is not likely to be incorporated into any cumulative patient data reports, graphs, or other data summarizing methods due to the data's inherent unreliability. The rules regarding data transfer of this nature may be configurable by any members of the system 100. Alternatively, default rules set by an administrator may be utilized. The client communication device 108 may then incorporate the transmitted patient information into a client-maintained patient record or alert the client user 120 to review the newly transmitted patient information to determine a health status of the patient 116.

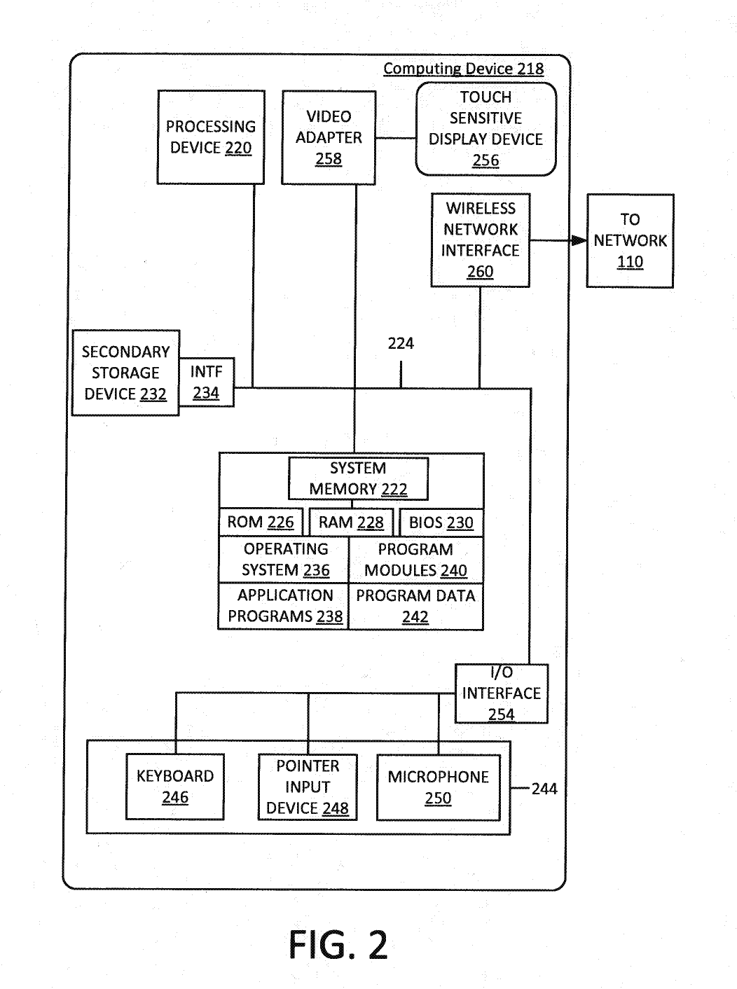

FIG. 2 illustrates an exemplary architecture of a computing device that can be used to implement aspects of the present disclosure, including the patient monitoring apparatuses 102, the central processing units 104, the healthcare communication device 106, the client communication device 108, and will be referred to herein as the computing device 218. The computing device 218 is used to execute the operating system, application programs, and software modules, as described herein.

The computing device 218 includes, in some embodiments, at least one processing device 220, such as a central processing unit (CPU). A variety of processing devices are available from a variety of manufacturers, for example, Intel® or Advanced Micro Devices. In this example, the computing device 218 includes a system memory 222, and a system bus 224 that couples various system components including the system memory 222 to the processing device 220. The system bus 224 is one of any number of types of bus structures including a memory bus, a memory controller, a peripheral bus, and a local bus using any of a variety of bus architectures.

Examples of computing devices suitable for the computing device 218 include a desktop computer, a laptop computer, a tablet computer, a mobile phone device such as a smart phone, or other devices configured to process digital instructions.

The system memory 222 includes read only memory (ROM) 226 and random access memory (RAM) 228. A basic input/output system (BIOS) 230, which contains the basic routines that act to transfer information within computing device 218, is typically stored in the ROM 226.

In some embodiments, the computing device 218 also includes a secondary storage device 232 such as a hard disk drive. Examples of hard disk drives include magnetic and solid state drives for storing digital data. The secondary storage device 232 is connected to the system bus 224 by a secondary storage interface (INTF) 234. The secondary storage device 232 and their associated computer readable media provide nonvolatile storage of computer readable instructions (including application programs and program modules), data structures, and other data for the computing device 218.

Although the exemplary environment described herein employs a hard disk drive as a secondary storage device, other types of computer readable storage media are used in other embodiments. Examples of these other types of computer readable storage media include magnetic cassettes, flash memory cards, digital video disks, Bernoulli cartridges, compact disc read only memories, digital versatile disk read only memories, random access memories, or read only memories. Some embodiments include non-transitory media.

A number of program modules can be stored in secondary storage device 232 or memory 222, including an operating system 236, one or more application programs 238, other program modules 240, and program data 242.

In an exemplary embodiment, the data stored in program data 242 can be represented in one or more files having any format usable by a computer. Examples include text files formatted according to a markup language and having data items and tags to instruct computer programs and processes how to use and present the data item. Examples of such formats include html, xml, and xhtml, although other formats can be used. Additionally, the data can be represented using formats other than those conforming to a markup language.

In some embodiments disclosed herein, findings are stored as data items in one or more data records. In some embodiments, data records are a set of one or more data items, such as in a format that can be read by a computing device. An example embodiment is a database record. Other examples of data records include tables, text files, computer executable files, data structures, or other structures for associating data items.

In some embodiments, computing device 218 includes input devices. Examples of input devices 244 include a keyboard 246, pointer input device 248, microphone 250, and touch sensitive display 256. Other embodiments include other input devices 244. The input devices are often connected to the processing device 220 through an input/output interface 254 that is coupled to the system bus 224. These input devices 244 can be connected by any number of input/output interfaces, such as a parallel port, serial port, game port, or a universal serial bus. Wireless communication between input devices and interface 254 is possible as well, and includes infrared, BLUETOOTH® wireless technology, 802.11a/b/g/n, cellular, or other radio frequency communication systems.

In some embodiments, a touch sensitive display device 256 is also connected to the system bus 224 via an interface, such as a video adapter 258. The touch sensitive display device 256 includes touch sensors for receiving input from a user when the user touches the display. Such sensors can be capacitive sensors, pressure sensors, or other touch sensors. The sensors not only detect contact with the display, but also the location of the contact and movement of the contact over time. For example, a user can move a finger or stylus across the screen to provide written inputs. The written inputs are evaluated and, in some embodiments, converted into text inputs. It is understood that all user selections described herein may be conducted by utilizing a finger to select or move an item on the touch sensitive display device 256. The touch sensitive display can use various different technologies such as resistive, surface acoustic wave, capacitive, infrared grids, projected optical imaging, dispersive signaling, and any other suitable touch technology. User interfaces displayed on the touch sensitive display device 256 can be operated with other types of input devices such as a mouse, touchpad, or keyboard. Other embodiments can use a non-touch display that is operated with an input device such as a mouse, touchpad, keyboard, or other type of input device. An IVR system or module may also be included or the functionality also provided, for example as one of the program modules 240, to facilitate interactions with a user, such as a patient or health care professional.

In addition to the display device 256, the computing device 218 can include various other peripheral devices (not shown), such as speakers or a printer.

When used in a local area networking environment or a wide area networking environment (such as the Internet), the computing device 218 is typically connected to the network 110 through a network interface, such as a wireless network interface 260. Other possible embodiments use other communication devices. For example, some embodiments of the computing device 218 include an Ethernet network interface, or a modem for communicating across the network 110.

The computing device 218 typically includes at least some form of computer-readable media. Computer readable media includes any available media that can be accessed by the computing device 218. By way of example, computer-readable media include computer readable storage media and computer readable communication media.

Computer readable storage media includes volatile and nonvolatile, removable and non-removable media implemented in any device configured to store information such as computer readable instructions, data structures, program modules or other data. Computer readable storage media includes, but is not limited to, random access memory, read only memory, electrically erasable programmable read only memory, flash memory or other memory technology, compact disc read only memory, digital versatile disks or other optical storage, magnetic cassettes, magnetic tape, magnetic disk storage or other magnetic storage devices, or any other medium that can be used to store the desired information and that can be accessed by the computing device 218.

Computer readable communication media typically embodies computer readable instructions, data structures, program modules or other data in a modulated data signal such as a carrier wave or other transport mechanism and includes any information delivery media. The term “modulated data signal” refers to a signal that has one or more of its characteristics set or changed in such a manner as to encode information in the signal. By way of example, computer readable communication media includes wired media such as a wired network or direct-wired connection, and wireless media such as acoustic, radio frequency, infrared, and other wireless media. Combinations of any of the above are also included within the scope of computer readable media.

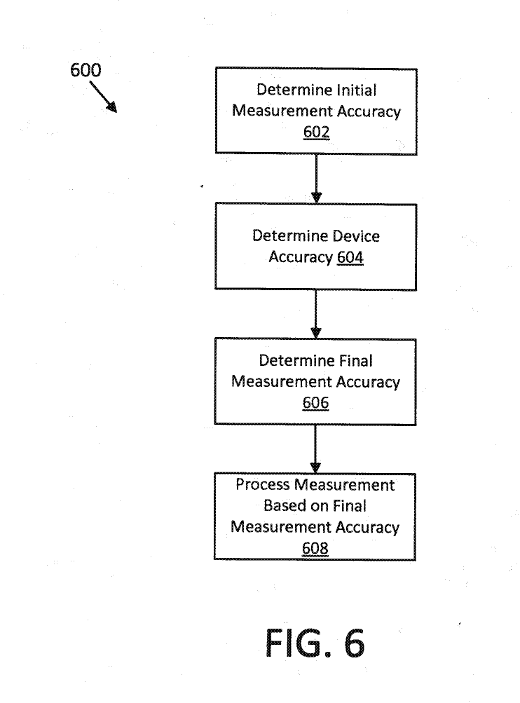

FIG. 6 is an embodiment of a general method 600 for assigning a probability of accuracy to an individual measurement received from a (patient monitoring) device. In the embodiment, the measurement is assigned or otherwise associated with a device-generated sampling date and time. The sampling date and time may be provided as metadata associated with the measurement and the metadata and measurement may be communicated as a unit depending on the protocol used. The sampling date and time is the internal time as maintained by the device at the instant that the measurement is taken. Because measurements need not be taken at the same time that the measurements are communicated to another system such as patient monitoring apparatus or central processing unit, this discussion will distinguish between the sampling date and time of the measurement and the current device date and time, which is the current time maintained by the device, and the system date and time, which is a date and time maintained by the accuracy testing system and used as a reference.

In the embodiment shown, the method 600 first assigns the measurement an initial measurement accuracy in an initial accuracy operation 602. In an embodiment, the initial measurement accuracy is determined based on the sampling date and time, and information known to the accuracy testing system performing the analysis. This information may include a stored system date and time of the previous communications with the device, the system date and time when the measurement was received from the device, and the current device date and time when the measurement was received from the device.

Method 600 further includes a device accuracy assessment operation 604 in which the device is assigned a device accuracy. In one embodiment, the device accuracy assessment operation 604 determines the device accuracy based on the current date and time of the device and the current date and time of the system. In one embodiment, the device accuracy assessment operation 604 determines the device accuracy based on the system date and time when the measurement was received from the device and the current device date and time when the measurement was received from the device.

Method 600 then uses the initial measurement accuracy and the device accuracy to determine a final measurement accuracy in a final accuracy assessment operation 606. For example, in an embodiment, the initial accuracy of a measurement may be identified by one of two possible categories: high likelihood or chance of accuracy and low likelihood or chance of accuracy. Likewise, the device accuracy may also be identified by one of two possible categories: high likelihood or chance of accuracy and low likelihood or chance of accuracy. Different combinations of these categorizations may be used to assign a final measurement accuracy to a measurement, which may also be identified as a category. For example, measurements for which the initial accuracy is high and its device accuracy is high may be assigned to a highest accuracy category, measurements for which the initial accuracy is high and its device accuracy is low may be assigned to a second highest accuracy category, measurements for which the initial accuracy is low and its device accuracy is high may be assigned to the second lowest accuracy category, measurements for which both the initial accuracy and its device accuracy are low may be assigned to the lowest accuracy category, and so on. After the final measurement accuracy is determined, the final measurement accuracy is then stored and otherwise associated with the measurement for further use when using the measurement in future analyses.

After the final measurement accuracy is determined in operation 606, the method 600 further includes a post-processing operation 608 in which the measurement is processed, handled, or otherwise used in later analyses based on its associated final measurement accuracy. As described above, there are many examples of how measurements may be used, screened or displayed based on the final measurement accuracy. In one embodiment, measurements with less than the highest accuracy may be presented to a user for manual analysis of whether to include the measurement in a patient record or not. Measurements with the highest accuracy, on the other hand, may be automatically displayed and added to patient records without human intervention. Automated analyses may be performed based on the final accuracy of entire sets of measurements for a patient in order to further identify which measurements should be automatically recorded as true measurements.

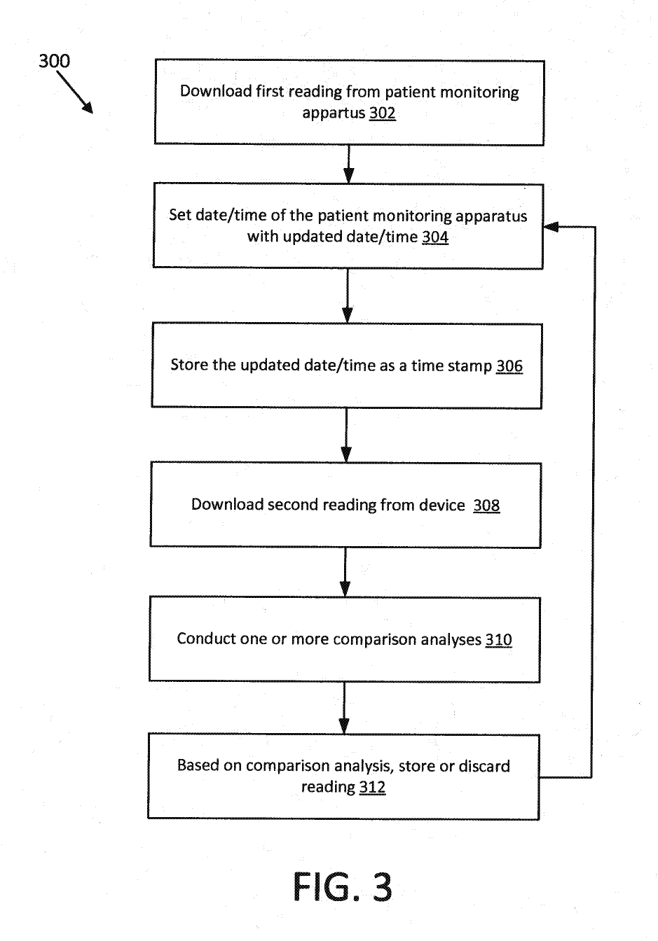

FIG. 3 is one example of a method 300 for testing the time accuracy of patient data. The method 300 is an exemplary embodiment of a method carried out by the accuracy testing system 112.

The method 300 begins at operation 302. At operation 302, the central processing unit 104 downloads a first reading or a set of readings from the patient monitoring apparatus 102. In the example, the first reading could be any data taken from a patient including, but not limited to, readings of blood pressure, blood sugar, heart rate, weight, blood oxygen, pain measurement, other biometrics, or patient answers to health-related questions. In some embodiments, the first reading may be temporarily stored in the central processing unit 104 so that the accuracy testing system 112 may analyze it for potential accuracy concerns.

In an embodiment, the readings may include a patient identifier, an apparatus identifier, or some other identification information that can be used to distinguish the data as being associated with a particular patient or device. The readings may further include the actual or modified data from the monitoring apparatus 102, patient input text or recorded sound or video data, or identifications of patient selected answers or inputs from a question and answer session. The readings may further include date and time information associated with data the being transmitted. The date and time information may include a separate date and time associated with each specific item of data (e.g., date and time associated with each blood pressure measurement, each answer, each heart rate measurement, etc.) and a date and time of any other event related to the patient monitoring. The date and time associated with an item of data may be generated by the monitoring device or by the patient monitoring apparatus. In addition, multiple date and times from different devices may be associated with an item of data and transmitted as part of the download of readings to the central processing unit 104.

The method 300 proceeds to operation 304, at which the accuracy testing system 112 sets the date and time of the patient monitoring apparatus 102 with an updated date and time. This also may include resetting the date and time, or directing the patient monitoring apparatus to reset the date and time, on all the ancillary monitoring devices from which the patient monitoring apparatus 102 collects patient data. In this way, the accuracy testing system 112 ensures that the date and time of the accuracy testing system 112 is the same as the date and time of the patient monitoring apparatus 102. In this operation 300, the current date and time of the patient monitoring apparatus 112, before it is reset, may be retrieved and stored. If this is the first use of the patient monitoring apparatus 102, the retrieved current date and time may be irrelevant or may be used to indicate that this is the first use of the apparatus.

Thereafter, the method 300 proceeds to operation 306, at which the accuracy testing system 112 stores the updated date and time. This serves as a first reference point to analyze the accuracy of the time stamp of patient data. The stored, updated date and time may be stored, at least temporarily, in the central processing unit 104.

Optionally, the patient monitoring apparatus 102 is disconnected from the system for a second reading to be taken.

Thereafter, the method 300 proceeds to operation 308, at which the accuracy testing system 112 downloads a second reading taken since updating the date and time of the patient monitoring apparatus 102 at operation 304. The one or more reading may also be stored, at least temporarily, in the central processing unit 104. At this point, the accuracy testing system 112 has access to readings taken prior to and after the date and time update.

Optionally, either before or after operation 308, the device accuracy is determined by comparing the date and time of the device (e.g., the patient monitoring apparatus) and the date and time of the system (e.g., the accuracy testing system).

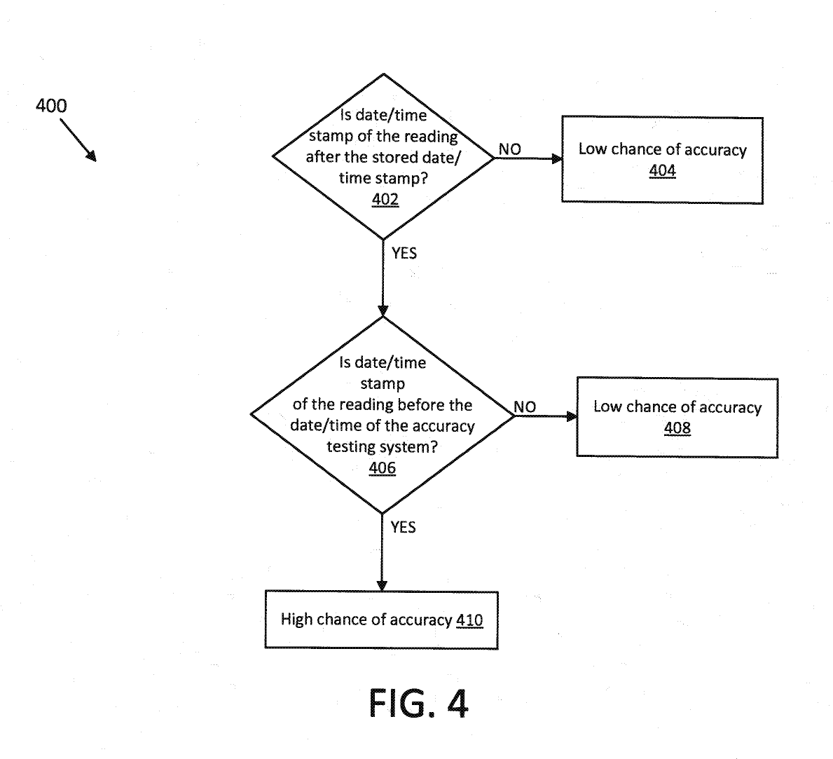

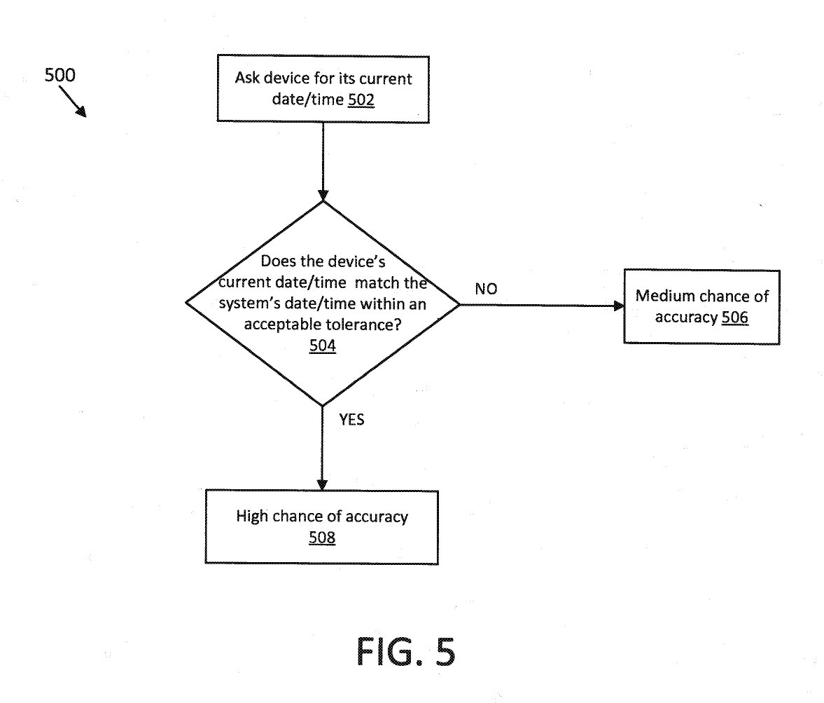

At operation 310, the accuracy testing system 112 conducts one or more comparison analyses based on the date and time stamp of readings taken from the patient monitoring apparatus 102. For example, the accuracy testing system 112 may make one or more comparisons between the date and time of the second readings with the stored date and time stamp in the system. Based on the results of the various tests, which are described in more detail in FIGS. 4 and 5, the accuracy testing system 112 determines the accuracy of one or more of the readings. For example, the accuracy testing system 112 may consider the reading to have a high chance of accuracy, a low chance of accuracy, or a medium chance of accuracy.

In one embodiment, the result of each test (e.g., method 400 in FIG. 4 and method 500 in FIG. 5) may be assigned a level of accuracy such as a high, low, or medium chance of accuracy. In one embodiment, the results of multiple tests can be combined and assigned a level of accuracy. For example, a combination of high chance of device accuracy and high chance of initial accuracy may be assigned the highest accuracy, a combination of low chance of device accuracy and high chance of initial accuracy may be assigned high accuracy, a combination of high chance of device accuracy and low chance of initial accuracy may be assigned low accuracy, and a combination of low chance of device accuracy and low chance of initial accuracy may be assigned lowest accuracy. Optionally, numerical values can be assigned to the various levels of accuracy. For example, the highest, high, low, and lowest accuracy described above may correspond to accuracy levels of 10, 8, 5, and 2, respectively. These are examples of how accuracy could be differentiated and the reader will understand that more or fewer gradations of accuracy may be used to categorize readings as desired.

At operation 312, and based on a pre-determined set of rules and the results at operation 310, the accuracy testing system 112 will either discard or store the readings. In one embodiment, operation 312 may include categorizing each item of data, readings, or portions of readings into different accuracy categories based on the results of the comparisons done in operation 310.

The rule set can be pre-determined as needed. For example, in some embodiments, the system may automatically accept readings with accuracy at or above a certain level (e.g., high chance of accuracy or an assigned numerical value of 8 out of 10) and/or remove readings with accuracy below certain level (e.g., lowest chance of accuracy or an assigned numerical value of 2 out of 10). In some embodiments, the accuracy testing system 112 may be required to discard all readings considered to have a low chance of accuracy. Additionally or alternatively, the accuracy testing system 112 may be required to store all readings considered to have a high chance of accuracy. In some embodiments, readings with a medium or low chance of accuracy (e.g., with an assigned numerical of below 8) may be stored for further review by a user such as a health care professional, who can discard, change, or accept the readings, or may undergo further testing by the central processing unit 104 or the accuracy testing system 112. Optionally, the accuracy assessment result is shown together with the readings to the user. In one embodiment, all readings and the corresponding accuracy assessment results are stored in the record for evaluation at a later date or informational purposes.

After the completion of operation 312, the method 300 may again reset the date and time stamp for the patient monitoring apparatus 102, prior to downloading further patient readings.

FIG. 4 is a flow chart illustrating an exemplary embodiment of a method 400 of testing the time accuracy of patient data. The method 400 is one example of a comparison analysis conducted by the accuracy testing system 112 during operation 310 in FIG. 3.

The method 400 begins at operation 402. At operation 402, the accuracy testing system 112 conducts one or more comparison analyses. It does so by analyzing the date and time of readings taken from a patient monitoring apparatus 102. For example, the accuracy testing system 112 compares the date and time of a reading with a stored date and time stamp for that apparatus. If the reading was taken after the date and time of a patient monitoring apparatus was updated and stored, then the reading should have a later date and time than the date and time of the update.

If the accuracy testing system 112 determines that the date and time of the reading is at a time before the stored date and time stamp, the method proceeds to operation 404, in which the accuracy testing system 112 categorizes the reading as having a low chance of date and time accuracy.

If the accuracy testing system 112 determines that the date and time of the reading occurs at a time after the updated date and time stamp, the method proceeds to operation 406. At operation 406, the accuracy testing system 112 assesses whether the date and time of the reading occurs before the current date and time stamp of the accuracy testing system 112. If the reading was taken earlier, the reading should have a date and time earlier than the current date and time of the accuracy testing system 112.

If the accuracy testing system 112 determines that the date and time of the reading does not occur before the current date and time stamp of the accuracy testing system 112, the method proceeds to operation 408. At operation 408, the accuracy testing system 112 categorizes the reading as having a low chance of date and time accuracy.

If the accuracy testing system 112 determines that the date and time of the reading occurs before the current date and time stamp of the accuracy testing system 112, the method proceeds to operation 410. At operation 410, the accuracy testing system 112 identifies the reading as having a high chance of date and time accuracy.

FIG. 5 is a flow chart illustrating an exemplary embodiment of a method 500 of testing the time accuracy of patient data. More specifically, the method 500 is another example of a comparison analysis conducted by the accuracy testing system 112 during operation 308 in FIG. 3. The method 500 may be performed in addition to the method 400 or as an alternative analysis.

The method 500 begins at operation 502, in which the current date and time of a patient monitoring apparatus is taken. At operation 504, to determine the likelihood of accuracy of a reading or a set of readings downloaded from the patient monitoring apparatus, the accuracy testing system 112 compares the date and time of a patient monitoring apparatus to the date and time of a system. This comparison can be performed at any time when the patient monitoring apparatus date/time and system date/time are simultaneously (or substantially simultaneously) available. Alternatively, the two date/times may be sampled at substantially the same time and stored for later comparison, which allows the method 500 to be performed after completion of a download as part of the post-processing of the patient data. It is understood that the system can be any system with a reference date and time. In this particular embedment, the system is the accuracy testing system 112. For example, the current date and time of the patient monitoring apparatus 102 is compared to the date and time of the accuracy testing system 112 to categorize the accuracy of the data.

More specifically, the current date and time of the patient monitoring apparatus 102 is compared to the date and time of the accuracy testing system 112 obtained at the same instant and it is determined whether it falls within an acceptable tolerance of the date and time of the accuracy testing system 112.

If the current date and time of the patient monitoring apparatus 102 is not within an acceptable tolerance of the date and time of the accuracy testing system 112, then the method proceeds to operation 506. In an embodiment, the acceptable tolerance may be specified by an operator of the system 100 as a range of times around the reference time. For example, it can be set that the patient monitoring apparatus date and time must be within 0.5 seconds of the reference date and time to be considered highly accurate. Multiple tolerances may be used, one for each category, for example. In addition, a tolerance range needs not be symmetrical around the reference time, e.g., the tolerance range may be between 5 seconds before the reference date/time and 1 second after the reference date/time. At operation 506, the accuracy testing system 112 categorizes the reading as being in a category associated with falling outside of the tolerance range, which in the embodiment shown is a medium chance of date and time accuracy category.

However, if the current date and time of the patient monitoring apparatus 102 is within an acceptable tolerance of the date and time of the accuracy testing system 112, then the method proceeds to operation 508. At operation 508, the accuracy testing system 112 determines that there is a high chance of date and time accuracy and associates the reading with that category.

It is understood that the comparison analyses described in FIGS. 4 and 5 may both be conducted by the accuracy testing system 112. However, in alternate embodiments, only one of the comparison analyses may be utilized by the accuracy testing system 112.

In one embodiment, operation 602 in FIG. 6 corresponds to the method 400 in FIG. 4. For example, the initial accuracy can be determined by comparing the sampling date and time with the stored date and time stamp (e.g., the system date and time of the previous communication with the device) and with the system date and time when the measurement was received from the device. It is understood that the system can be any system with a reference date and time, including the accuracy testing system 112.

In one embodiment, operation 604 in FIG. 6 corresponds to the method 500 in FIG. 5. For example, the device accuracy can be determined by comparing the current date and time of the device when the measurement was received from the device with the system date and time when the measurement was received from the device. It is understood that the system can be any system with a reference date and time, including the accuracy testing system 112.

As described above, the methods of FIGS. 4 and 5 and the operations of FIG. 6 may be performed independently or together. For example, the methods may be combined so that all readings that are categorized as having a high chance of accuracy in operation 410 of FIG. 4 are further analyzed using the method 500 of FIG. 5 so that some readings may be recategorized as having a medium chance of accuracy.

Also described above, based on the categorization readings may be stored, analyzed, or presented differently. In one embodiment, only high chance of accuracy data may be automatically stored and displayed to a health care provider. Less accurate data may displayed differently, may not be automatically made a part of the patient's record, or may not be displayed but are nonetheless accessible (e.g., the display identifies to the healthcare provider that less accurate readings exist but are not displayed). In one embodiment, if the device accuracy is low, the system may prevent further measurement. Measurement may take place when the device accuracy is within an acceptable tolerance.

Additional follow-up actions may also be performed based on data categorizations. For example, an alert or notification may be transmitted to the health care provider is too many readings are received in lower accuracy categories within a given amount of time. For example, receiving more than a threshold number of readings in a specific category over a period of time (e.g., more than 10 highly inaccurate readings in a month) may trigger an intervention by the health care provider to determine if the patient monitoring device needs to be replaced or an automatic notification to replace the device.

In addition, a determination made in real time (that is, during a communication session directly with the patient via the patient monitoring apparatus) that a downloaded reading is likely to be inaccurate may result in an immediate request that the patient redo the measurement or measurements determined to be inaccurate.

An alternative embodiment of the determination system and method can be implemented on the patient monitoring apparatus 102. In this embodiment, an accuracy testing module (not shown) that can serve the function of the accuracy testing system 112 is incorporated into the patient monitoring apparatus 102 to assesses the accuracy of patient data received from one or more monitoring peripheral 101. In this case, the monitoring peripherals 101 can serve the function of the patient monitoring apparatus 102 in the previously-described embodiments. As in the embodiments above, the accuracy of each measurement from the monitoring peripheral 101 is assessed based on a comparison of the current date and time of the monitoring peripheral 101, the current date and time of the patient monitoring apparatus 102, the date and time associated with the measurement taken by the device, and the date and time of the last time the monitoring peripheral 101 communicated with the patient monitoring apparatus 102.

In this embodiment, the method of FIG. 3 is performed by the patient monitoring apparatus 102 as part of the interactions with the one or more monitoring peripheral. Thus, in one embodiment, in operation 302, a measurement is obtained from a given peripheral 101, along with any desired identifiers, a date and time for the measurement as assigned by the peripheral 101 as well as the current date and time as maintained by the peripheral 101. In operation 304, the patient monitoring apparatus 102 resets the peripheral's date and time with the current apparatus date and time and a record that this updating of the peripheral's internal clock is recorded in operation 306. Later, a second measurement is obtained or downloaded to the patient monitoring apparatus 102 from the peripheral in operation 308. Again, this includes receiving the measurement, and, depending on the embodiment, a date and time of the measurement as assigned by the peripheral and the current date and time as maintained by the peripheral's internal clock. Comparisons, such as those described in FIGS. 4 and 5, may then be performed in operation 310. Operation 312 may then categorize and subsequently store or discard any measurements.

In addition, in all of the embodiments described, the categorization information may be transmitted along with the measurements to the central processing unit 104 via the network as part of the patient monitoring apparatus's normal data reporting process. In this embodiment, each reported measurement may be associated with some identifier that is indicative of the likelihood of accuracy categorization of that measurement. Thus, the reported data may include such information as a) the measurement; b) an identifier of the patient monitoring apparatus or peripheral that was the source of the measurement; c) the date and time associated with the measurement by the patient monitoring apparatus or peripheral; and d) an identifier of the probability of accuracy category assigned to the measurement by the accuracy testing system or the accuracy testing module on the patient monitoring apparatus. In addition, any other information described herein may be included depending on how detailed a record is desired to be maintained at the central processing unit 104.

In addition to the testing described above, other factors may be used to determine the accuracy category by the accuracy testing system 112 or the accuracy testing module on the patient monitoring apparatus 102. For example, if the accuracy testing module on the patient monitoring apparatus 102 determines that a peripheral 101 (or, in the case of a separate accuracy testing system, the accuracy testing system 112 determines that the patient monitoring apparatus 102) has suffered a loss of power, battery failure, memory failure, unexpected or expected reboot of the peripheral's firmware, time reset, manual modification of date and time, or any other event that could comprise the date and time since the previous communication with the apparatus 102 (or, in the case of a separate accuracy testing system, since the previous communication of the patient monitoring apparatus 102 with the accuracy testing system 112), then the accuracy testing module or system may automatically assign a low accuracy category to the measurement regardless of the outcome or in lieu of the methods of FIGS. 3-5. Alternatively, this information may be used to otherwise lower the accuracy category of the measurement by some amount.

Probability of accuracy categories may be assigned numbers and may also be presented as a range or spectrum. For example, the probably of accuracy may be a score ranging from a maximum of 10 (indicating a likelihood that the measurement or readings is highly accurate) to a minimum of 1 (indicating that the measurement is, in all likelihood, is very inaccurate).

The various embodiments described above are provided by way of illustration only and should not be construed to limit the claims attached hereto. Those skilled in the art will readily recognize various modifications and changes that may be made without following the example embodiments and applications illustrated and described herein, and without departing from the true spirit and scope of the following claims.

Embodiment 1 encompasses a method for reviewing data, the method comprising:
receiving, at a computing device, a first patient reading from a remote patient device, the first patient reading including first patient data and at least one first date and time associated with the first patient data;
storing, at the computing device, the first patient reading in a database;
updating, from the computing device, time and date settings of the remote patient device to a then-current value of a reference time;
receiving, at the computing device, a second patient reading from the remote patient device, the second patient reading including second patient data and at least one second date and time associated with the second patient data; and
conducting, at the computing device, one or more comparison analyses on second date and time and then-current value of the reference time to categorize the accuracy for at least one of the first and second patient readings, wherein the one or more comparison analyses are based on a predefined rule set.

Embodiment 2 encompasses the method of embodiment 1, wherein the predefined rule set is configurable by at least one of a client and a user associated with the client.

Embodiment 3 encompasses the method of embodiment 1, wherein the predefined rule set is configurable by at least one of the health care communication device and the health care professional.

Embodiment 4 encompasses the method of embodiment 1, further comprising:
storing, at the computing device, an updated date and setting as a date and time stamp;
assessing, at the computing device, whether the second date and time of the second patient reading is after the time and date stamp; and
assessing, at the computing device, whether the date and time of the first and second patient readings are before a date and time settings of the computing device.

Embodiment 5 encompasses the method of embodiment 1, further comprising:
assessing, at the computing device, whether the date and time settings of the remote patient device match a time and date settings of the computing device, wherein the match falls within an acceptable tolerance.

Embodiment 6 encompasses the method of embodiment 1, further comprising:
storing, at the computing device, an updated date and time setting as a date and time stamp;
assessing, at the computing device, whether a date and time of the first and second patient reading is after the date and time stamp;
assessing, at the computing device, whether the date and time of the first and second patient reading is before a date and time settings of the computing device; and
assessing, at the computing device, whether the date and time settings of the remote patient device match a date and time settings of the computing device, wherein the match falls within an acceptable tolerance.

Embodiment 7 encompasses the method of embodiment 1, further comprising:
discarding the first patient reading or the second patient reading, when the one or more comparison analyses indicates that the first patient reading or the second patient reading has a low chance of accuracy.

Embodiment 8 encompasses the method of embodiment 1, wherein the first patient reading includes at least one of: a blood pressure measurement, a blood glucose measurement, an ECG measurement, a weight measurement, a pulse oxygen measurement, a blood oxygen measurement, a heart rate measurement, a heart rhythm measurement, and a peak flow measurement.

Embodiment 9 encompasses the method of embodiment 1, wherein at least one of the first patient reading and the second patient reading includes patient answers to health-related questions.

Embodiment 10 encompasses the method of embodiment 1, wherein the second patient reading includes a plurality of patient readings.

Embodiment 11 encompasses a system for reviewing data, the system comprising:
a remote patient monitoring apparatus; and
a central processing unit, the central processing unit including an accuracy testing system and a database storing a predefined rule set, the accuracy testing system in communication with the remote patient monitoring apparatus via a network,

wherein the remote patient monitoring apparatus is configured to:
receive a first patient reading from a remote patient device;
store the first patient reading in a database;
update date and time settings of the remote patient device;
receive a second patient reading from the remote patient device; and
conduct one or more comparison analyses on the first and second patient readings to determine an accuracy for each of the first and second patient readings,

wherein the one or more comparison analyses are based on a predefined rule set;

and wherein the accuracy testing system is configured to:
store an updated date and time setting as a time and date stamp;
assess whether a date and time of the first and second patient reading is after the time and date stamp;
assess whether the date and time of the first and second patient reading is before a date and time settings of the computing device; and
assess whether the date and time settings of the remote patient device match a time and date settings of the computing device, wherein the match falls within an acceptable tolerance.

Embodiment 12 encompasses the system of embodiment 11, wherein the system comprises a plurality of remote patient monitoring apparatuses.

Embodiment 13 encompasses a computer-readable storage device that includes a computer-executable instruction that employ a method for reviewing data, the instruction comprising:
receiving, at a computing device, a first patient reading from a remote patient device;
storing, at the computing device, the first patient reading in a database;
updating, from the computing device, date and time settings of the remote patient device;
receiving, at the computing device, a second patient reading from the remote patient device; and
conducting, at the computing device, one or more comparison analyses on the first and second patient readings to determine an accuracy for each of the first and second patient readings, wherein the one or more comparison analyses are based on a predefined rule set.

Embodiment 14 encompasses the system of embodiment 13, wherein the predefined rule set is configurable by at least one of a client and a user associated with the client.

Embodiment 15 encompasses the system of embodiment 13, wherein the predefined rule set is configurable by at least one of the health care communication device and the health care professional

Embodiment 16 encompasses the system of embodiment 13, the instruction further comprising:
storing, at the computing device, an updated date and time setting as a date and time stamp;
assessing, at the computing device, whether a date and time of the first and second patient reading is after the date and time stamp; and
assessing, at the computing device, whether the date and time of the first and second patient reading is before a date and time settings of the computing device.

Embodiment 17 encompasses the system of embodiment 13, the instruction further comprising:
assessing, at the computing device, whether the date and time settings of the remote patient device match a date and time settings of the computing device, wherein the match falls within an acceptable tolerance.

Embodiment 18 encompasses the system of embodiment 13, the instruction further comprising:
storing, at the computing device, an updated date and time setting as a date and time stamp;
assessing, at the computing device, whether a date and time of the first and second patient reading is after the time and date stamp;
assessing, at the computing device, whether the date and time of the first and second patient reading is before a date and time settings of the computing device; and
assessing, at the computing device, whether the date and time settings of the remote patient device match a date and time settings of the computing device, wherein the match falls within an acceptable tolerance.

Embodiment 19 encompasses the system of embodiment 13, the instruction further comprising:
discarding the first patient reading or the second patient reading, when the one or more comparison analyses indicates that the first patient reading or the second patient reading has a low chance of accuracy.

Embodiment 20 encompasses the system of embodiment 13, the instruction further comprising:
marking the first patient reading or the second patient reading as high accuracy, when the one or more comparison analyses indicates that the first patient reading or the second patient reading has a high chance of accuracy.

What is claimed is:

1. A method for processing patient data, the method comprising:
(a) updating the date and time of a patient monitoring apparatus;
(b) storing the updated the date and time of the patient apparatus;
(c) receiving a patient reading, the patient reading including patient data and at least one apparatus date and time associated with the patient data;
(d) conducting one or more comparison analyses on the stored updated date and time, the current date and time, and the patient reading to determine the time accuracy of the patient data; and
(e) assigning an accuracy assessment value to the patient reading.

2. The method of claim 1, wherein the patient monitoring apparatus comprises an accuracy testing module for performing step (d).

3. The method of claim 2, further comprising:
receiving the patient reading from one or more monitoring peripherals.

4. The method of claim 1, wherein step (d) comprises:
(d1) assessing whether the apparatus date and time associated with the patient data is after the stored date and time; and
(d2) assessing whether the apparatus date and time associated with the patient data is before the current date and time.

5. The method of claim 1, further comprising:
assessing whether the apparatus date and time of the patient monitoring apparatus falls within an acceptable tolerance of a system date and time taken at the same time.

6. The method of claim 4, further comprising:
assessing whether the apparatus date and time of the patient monitoring apparatus falls within an acceptable tolerance of a system date and time taken at the same time.

7. The method of claim 1, further comprising:
discarding the patient data when the one or more comparison analyses indicates that the patient data has a low chance of accuracy.

8. The method of claim 1, wherein the patient reading includes at least one of:
a blood pressure measurement, a blood glucose measurement, an ECG measurement, a weight measurement, a pulse oxygen measurement, a blood oxygen measurement, a heart rate measurement, a heart rhythm measurement, and a peak flow measurement.

9. The method of claim 1, wherein the patient reading includes patient answers to health-related questions.

10. The method of claim 1, wherein the patient reading includes a plurality of patient readings.

11. A system for remote patient monitoring, the system comprising:
a patient monitoring apparatus; and
a central processing unit comprising an accuracy testing system,
wherein the accuracy testing system is configured to:
(a) update the date and time of the patient monitoring apparatus based on the system date and time of the accuracy testing system;
(b) store the updated the date and time of the patient monitoring apparatus;
(c) receive a patient reading from the patient monitoring apparatus, the patient reading including patient data and at least one apparatus date and time associated with the patient data;
(d) conduct one or more comparison analyses on its system date and time, the stored updated date and time, and the patient reading to determine the time accuracy of the patient data;
(e) assign an accuracy assessment value to the patient reading.

12. The system of claim 11, wherein the accuracy testing system is further configured to perform at least one of the following:
(d1) assessing whether the apparatus date and time associated with the patient data is after the stored date and time;
(d2) assessing whether the apparatus date and time associated with the patient data is before the current system date and time; and
(d3) assessing whether the apparatus date and time of the patient monitoring apparatus falls within an acceptable tolerance of the system date and time taken at the same time.

13. A computer-readable storage device comprising computer-executable instructions that employ a method for processing data, the instructions comprising:
(a) updating the date and time of a patient monitoring apparatus;
(b) storing the updated the date and time of the patient monitoring apparatus;
(c) receiving a patient reading, the patient reading including patient data and at least one apparatus date and time associated with the patient data;
(d) conducting one or more comparison analyses on the stored updated date and time, the current date and time, and the patient reading to determine the time accuracy of the patient data, and
(e) assigning an accuracy assessment value to the patient reading.

14. The computer-readable storage device of claim 13, wherein the patient monitoring apparatus comprises an accuracy testing module for performing step (d).

15. The computer-readable storage device of claim 13, wherein the instructions comprises:
receiving the patient reading from one or more monitoring peripherals.

16. The computer-readable storage device of claim 13, wherein the instructions further comprises:
(d1) assessing whether the apparatus date and time associated with the patient data is after the stored date and time; and
(d2) assessing whether the apparatus date and time of the patient data is before the current date and time.

17. The computer-readable storage device of claim 13, wherein the instructions further comprises:
assessing whether the apparatus date and time of the patient monitoring apparatus falls within an acceptable tolerance of a system date and time taken at the same time.

18. The computer-readable storage device of claim 13, wherein the instructions further comprises:
discarding the patient data when the one or more comparison analyses indicates that the patient data has a low chance of accuracy.

19. The computer-readable storage device of claim 13, wherein the patient reading includes at least one of: a blood pressure measurement, a blood glucose measurement, an ECG measurement, a weight measurement, a pulse oxygen measurement, a blood oxygen measurement, a heart rate measurement, a heart rhythm measurement, and a peak flow measurement.

20. The computer-readable storage device of claim 13, wherein the patient reading includes patient answers to health-related questions.
